(19) United States
(12) Patent Application Publication
(10) Pub. No.: US 2020/0375847 A1
(43) Pub. Date: Dec. 3, 2020

(54) ANESTHETICS NEEDLE DEVICE TO FACILITATE THE USE OF BUFFERED ANESTHETICS

(71) Applicants: Haley Lee Frame, Lynn, IN (US); Wesley Michael Frame, Lynn, IN (US)

(72) Inventors: Haley Lee Frame, Lynn, IN (US); Wesley Michael Frame, Lynn, IN (US)

(21) Appl. No.: 16/475,470

(22) PCT Filed: Jan. 3, 2018
(86) PCT No.: PCT/US2018/012189

§ 371 Date: Jul. 2, 2019

Related U.S. Application Data
(60) Provisional application No. 62/441,630, filed on Jan. 3, 2017.

(30) Foreign Application Priority Data
Jan. 3, 2018 (US) PCT/US2018/012189

(51) Int. Cl. A61J 1/20; A61J 1/14; A61J 1/06; A61M 39/10

(52) U.S. Cl. CPC A61J 1/2096; A61J 1/1406; A61M 2039/1033; A61M 39/10; A61J 1/062

(57) ABSTRACT

An anesthetic needle device having a buffer cartridge for storing and moving a buffer solution into a syringe having an anesthetic carpule; an exhaust cartridge for transferring a buffer solution into a syringe having an anesthetic carpule and also receiving displaced anesthetic from the anesthetic carpule; and a needle assembly for injecting a buffer solution into a syringe having an anesthetic carpule and also injecting a buffered-anesthetic into a patient.

### FIELD OF THE INVENTION

The present invention relates to methods and device for buffering anesthetics. More particularly, the invention relates to a needle assembly to be used in conjunction with industrial anesthetic carpule syringes to buffer anesthetic immediately before application of the anesthetic to a patient.

### BACKGROUND OF THE INVENTION

Local anesthetics are the safest and most effective drugs in medicine for the prevention and management of pain during medical and dental procedures. Anesthetics manage the pain by preventing the nociceptive impulse from reaching the brain. In an effort to prolong the shelf life of anesthetics, the anesthetic is often made acidic. Most, if not all, local anesthetic formulations are acidic, with a pH below 7.0 which makes it more shelf stable and result in an extended shelf life. Unfortunately, the acidic nature of typical anesthetics results in discomfort upon injection, delayed effectiveness of the anesthetic, post-injection tissue injury and unreliable effectiveness in the presence of infection and inflammation. All of these drawbacks can be improved with alkalizing anesthetics with buffer solution, such as sodium bicarbonate, to raise the pH to become more neutral prior to injection of the anesthetic.

Lidocaine HCl is one typical local anesthetic that may be chosen by a medical or dental practitioner for use for pain management with a patient. For lidocaine HCl, where the pH is 3.5, 99.99% of the solution is in the charged, or ionized cationic, form which is not lipid soluble. Anesthetic can only cross the nerve membrane when the body converts it to the uncharged, or deionized, free base, form as it approaches a more neutral pH (7.35-7.45). The time that this transformation takes place results in a latency period of the anesthetic. Buffering the acidic anesthetic with, for example, a bicarbonate buffer significantly increases the amount of the active deionized form. Raising the pH from about 3.5 to about 7.4 produces a 6000-fold increase in efficacy. Unfortunately, the addition of the buffering solution cannon be added well prior to the injection use, such as at the factory site or even in the practitioners' office hours or days in advance because of the reduction in shelf life of the product where the efficacy of the medication is significantly reduced at use and the end result if unsatisfactory. Consequently, it is important that a device allows for buffering of the local anesthetic just prior to use.

Syringes are used to deliver many medications, including anesthetic. For example, syringes are used to deliver anesthetic to patients before performing medical or dental work. Many people dislike receiving injections. Receiving injections may cause anxiety and discomfort for the person that extends beyond the pain associated with being pierced by a needle and receiving the injection. Dental and medical practitioners address this patient anxiety and discomfort in a variety of ways. In some instances, practitioners use two different anesthetics. A first anesthetic to numb the area even though the numbing effect may be short lived to the dilating effects of the first anesthetic. The first anesthetic may be used to numb the area sufficiently to allow the practitioner to inject a second anesthetic, which contains the lower pH vasoconstriction, long term anesthetic. However, patients who are afraid of injections generally are even less comfortable with two injections.

Alternately, practitioners may increase the pH of the anesthetic by the addition of buffer solution, such as sodium bicarbonate. Anesthetic is often delivered using disposable carpules placed into a durable syringe. As such, practitioners can increase the pH of the anesthetic by adding buffer solution directly into the anesthetic carpules, generally by injection as described, for example in U.S. Pat. No. 5,603,695. As the anesthetic carpules are manufactured being completely filled, the addition of a buffer solution requires the first removal of some anesthetic and then the subsequent addition of the buffer solution. This is a time-consuming process.

In another alternative approach, as exampled in U.S. Pat. No. 8,162,917, an independent buffering device may be used to deliver buffer solution directly into anesthetic carpules for subsequent use throughout the day with a durable syringe. In this approach, however, the same needle assembly is used to prepare a plurality of anesthetic carpules in the buffering device and thus presents sterility and potential cross-contamination issues. Further, this approach requires the removal of the buffering device (needle assembly and mixing chamber) and the subsequent attachment of a different injection needle for the injection of the buffered anesthetic solution into the patient. Further, this approach requires the anesthetic carpules to be isolated (i.e., not stored within the durable syringe). After the buffering, the anesthetic carpule must be inserted into the durable syringe and then subsequently the addition of an injection needle. This approach is time-consuming and also often results in the buffering of an excess number of carpules, and because buffered anesthetic solution has a limited shelf-life, they are often discarded by the end of the workday.

What is need is a method and device for the buffering of anesthetic, especially anesthetic contained in a syringe carpule, wherein the device provides a needle assembly adapted for dual-functionality of quick, single-patient buffering of anesthetic and also the subsequent injection of the buffered-anesthetic using the same needle assembly.

### SUMMARY OF THE INVENTION

The present invention is a dual-purpose anesthetic needle device which is intended to be attached to a medical/dental syringe already housing a carpule of local anesthetic, and just prior to use, provide a safe and sterile way to push buffer solution into the local anesthetic carpule. The syringe can then be taken directly to the site of injection. After use, the anesthetic needle device may be disposed of.

An anesthetic needle device may comprise a buffer cartridge for storing and moving a buffer solution into a syringe having an anesthetic carpule; an exhaust cartridge for transferring a buffer solution into a syringe having an anesthetic carpule and also receiving displaced anesthetic from the anesthetic carpule; and a needle assembly for injecting a buffer solution into a syringe having an anesthetic carpule and also injecting a buffered-anesthetic into a patient.

The needle assembly may comprise a transfer needle, said transfer needle being capable of transferring a buffer solution from said buffer cartridge and into the anesthetic carpule; an exhaust needle, said exhaust needle being capable of receiving the anesthetic which was displaced from the anesthetic carpule by the injection of the buffer solution; and a connector, said connector providing structural support for said transfer needle and said exhaust needle and also providing releasable-attachment of said needle assembly to the syringe.

The transfer needle and the exhaust needle may be coaxial to each other. Alternatively, the transfer needle and the exhaust needle may be non-coaxial to each other.

The exhaust cartridge may comprise an exhaust shell, said exhaust shell providing structural support for said exhaust cartridge; and an exhaust housing adapted to form an exhaust chamber for the receiving of anesthetic which was displaced from the anesthetic carpule by the injection of the buffer solution; wherein said transfer needle has a buffer-end and a syringe-end, said buffer-end being in fluid communication with buffer solution stored in the buffer cartridge, said syringe-end being in fluid communication with the anesthetic in the anesthetic carpule; wherein said exhaust needle has a buffer-end and a syringe-end, said buffer-end being in fluid communication with exhaust chamber, said syringe-end being in fluid communication with the anesthetic in the anesthetic carpule.

The buffer cartridge may comprise a plunger for actuating the movement of buffer solution; a plunger stem extending from said plunger; a piston connected to said plunger stem and being adapted to push the buffer solution when actuated by said plunger; and a buffer containment adapted to store the buffer solution and allowing for the movement of the piston.

Plunger markings may be placed on said plunger stem for assisting in the metering of the buffer solution.

The buffer cartridge may further comprise a septum for providing a protective membrane of said buffer containment until such time that said transfer needle is repositioned to pierce said septum for subsequent moving of the buffer solution.

The anesthetic needle device may further comprise a buffer solution stored in the buffer cartridge.

The connector may have threads being adapted for releasable-engagement with the syringe. Alternatively, the connector may be made of a rubber-like material for the force-fit, releasable-engagement with the syringe.

An anesthetic needle device may comprise a buffer cartridge for storing and moving a buffer solution into a syringe having an anesthetic carpule; an exhaust cartridge for transferring a buffer solution into a syringe having an anesthetic carpule and also receiving displaced anesthetic from the anesthetic carpule; a needle assembly for injecting a buffer solution into a syringe having an anesthetic carpule and also injecting a buffered-anesthetic into a patient; and a buffer solution stored in the buffer cartridge.

The needle assembly may comprise a transfer needle, said transfer needle being capable of transferring a buffer solution from said buffer cartridge and into the anesthetic carpule; an exhaust needle, said exhaust needle being capable of receiving the anesthetic which was displaced from the anesthetic carpule by the injection of the buffer solution; and a connector, said connector providing structural support for said transfer needle and said exhaust needle and also providing releasable-attachment of said needle assembly to the syringe.

The transfer needle and the exhaust needle may be coaxial to each other. Alternatively, the transfer needle and the exhaust needle may be non-coaxial to each other.

The exhaust cartridge may comprise an exhaust shell, said exhaust shell providing structural support for said exhaust cartridge; and an exhaust housing adapted to form an exhaust chamber for the receiving of anesthetic which was displaced from the anesthetic carpule by the injection of the buffer solution; wherein said transfer needle has a buffer-end and a syringe-end, said buffer-end being in fluid communication with buffer solution stored in the buffer cartridge, said syringe-end being in fluid communication with the anesthetic in the anesthetic carpule; wherein said exhaust needle has a buffer-end and a syringe-end, said buffer-end being in fluid communication with exhaust chamber, said syringe-end being in fluid communication with the anesthetic in the anesthetic carpule.

The buffer cartridge may comprise a plunger for actuating the movement of buffer solution; a plunger stem extending from said plunger; a piston connected to said plunger stem and being adapted to push the buffer solution when actuated by said plunger; and a buffer containment adapted to store the buffer solution and allowing for the movement of the piston.

The anesthetic needle device is designed for transferring a first solution into a syringe having a carpule containing a second solution and also for injecting a mixture of the first and second solutions into a patient. The device may comprise an exhaust housing providing structural support for said device; an exhaust chamber being formed by said exhaust housing; a transfer needle, having a first-end extending exterior to said exhaust housing and a second-end extending exterior to the exhaust needle, said transfer needle being capable of transferring a first solution into the carpule containing a second solution; an exhaust needle, having a first-end in communication with said exhaust chamber and a second-end extending short of said transfer needle second-end, said exhaust needle being capable of transferring a second solution out of the carpule and into said exhaust chamber; and a connector providing structural support for said transfer needle and said exhaust needle and also providing releasable-attachment of said device to the syringe. The transfer needle and the exhaust needle may be either coaxial or non-coaxial to each other.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Non-limiting and non-exhaustive examples of the present invention are described with reference to the following figures, wherein like reference numerals refer to like parts throughout the various views unless otherwise specified. None of the drawings are necessarily to scale.

FIG. 1 is a right-side elevational view of an anesthetics needle device in accordance with the present invention;

FIG. 2 is a perspective view of the anesthetics needle device in FIG. 1;

FIG. 3 is a perspective view of the anesthetics needle device in FIG. 2 with the protective cap being removed;

FIG. 4 is a right-side elevational view of the anesthetics needle device being aligned with a syringe having an anesthetic carpule inside;

FIG. 5 is a right-side elevational view of the anesthetics needle device being inserted into the syringe having an anesthetic carpule inside;

FIG. 6 is a cross-sectional view of the anesthetics needle device and syringe in FIG. 5;

FIG. 7A is a cross-sectional view of the anesthetics needle device in FIG. 2;

FIG. 7B is a cross-sectional view of the anesthetics needle device in FIG. 7A with the protective cap being removed;

FIG. 7C is a cross-sectional view of the anesthetics needle device in FIG. 7B with the needle assembly being attached to the syringe and carpule;

FIG. 7D is a cross-sectional view of the anesthetics needle device in FIG. 7C with the plunger being initially actuated in the direction of arrow A such that the transfer needle pierces the septum and becomes in fluid communication with the buffer solution;

FIG. 7E is a cross-sectional view of the anesthetics needle device in FIG. 7D with the plunger being further actuated in the direction of arrow A such that buffer solution is moved into the anesthetic carpule and contemporaneously a portion of the anesthetic is displaced through the exhaust needle into the exhaust chamber;

FIG. 7F is a cross-sectional view of the anesthetics needle device in FIG. 7E with the buffer cartridge and exhaust cartridge being removed and subsequently the needle assembly being made available for injection into the patient;

FIG. 7F is a cross-sectional view of the anesthetics needle device in FIG. 7E with the buffer cartridge and exhaust cartridge being removed and subsequently the needle assembly being made available for injection into the patient;

FIG. 7G is a cross-sectional view of the needle assembly in FIG. 7F showing the needle assembly being attached to the syringe with the carpule now containing buffered anesthetic;

FIG. 8A is a cross-sectional view of the needle assembly and syringe nozzle with the connector being shown having threads for secure releasable-attachment to the syringe;

FIG. 8B is a cross-sectional view of an alternate needle assembly with an alternate connector being made of a rubber-lime material for force-fit attachment to the syringe;

FIG. 9 is a cross-sectional view of an alternate needle assembly having an exhaust vent so that exhausted-anesthetic may enter the mixing chamber more easily;

FIG. 10 is a cross-sectional view of an alternate needle assembly wherein the transfer needle and the exhaust needle are non-coaxial;

FIG. 11 is a cross-sectional view of the plunger having plunger markings on the plunger stem so as assist in measuring the amount of buffered solution injected into the carpule;

FIG. 12 is an exploded view of FIG. 7 showing an exemplary construction of the anesthetic needle device;

FIG. 13A is a cross-sectional view of an alternate embodiment having only the needle assembly and exhaust chamber together; and

FIG. 13B is a cross-sectional view of an alternate embodiment having only the needle assembly and exhaust chamber, together with a syringe and a separate vial of buffer solution being used.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help improve understanding of various examples of the present invention. Also, common but well-understood elements that are useful or necessary in a commercially feasible embodiment are often not depicted in order to facilitate a less obstructed view of these various embodiments of the present invention.

It will be appreciated that the drawings are illustrative and non-limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION OF THE INVENTION

The anesthetic needle device of the present invention provides a dual-purpose injection needle which is intended to be attached to a medical/dental syringe already housing a carpule of the local anesthetic, and just prior to use, provide a timely, safe and sterile pathway to push buffer solution into the local anesthetic cartridge and excess anesthetic solution out, all by the advancement of the buffer solution's plunger. The syringe can then be taken directly to the site of injection.

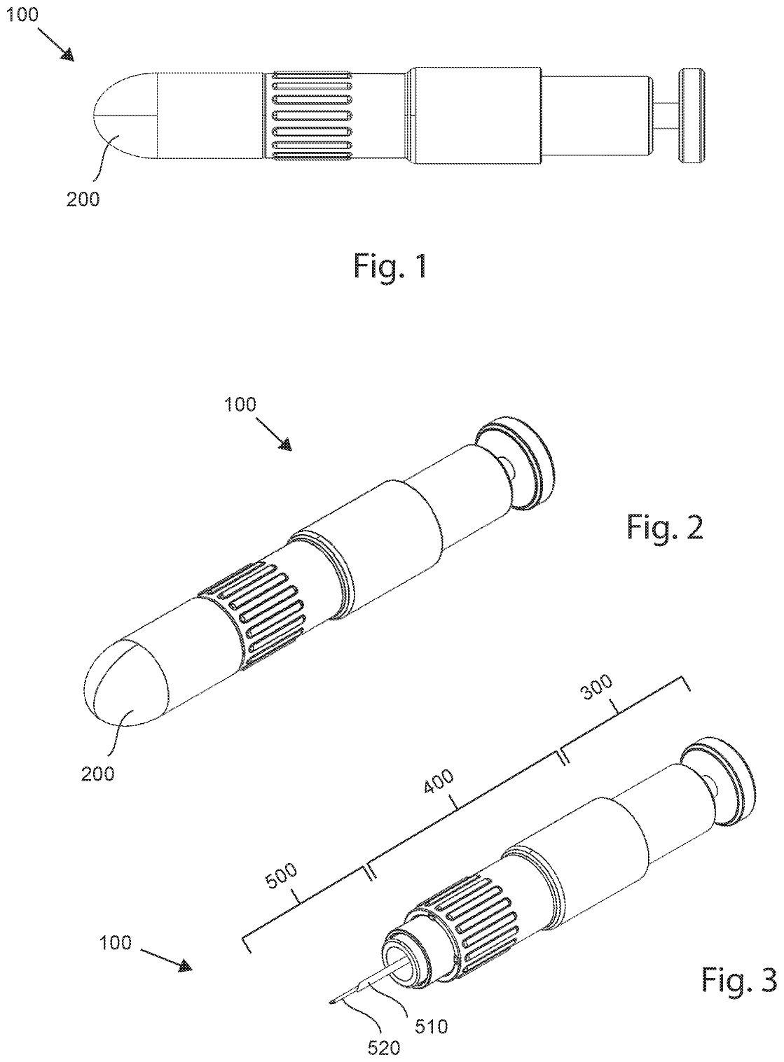

FIG. 1 shows is a right-side elevational view of an exemplary embodiment of an anesthetics needle device 100 in accordance with the present invention. In this initial view, a protective cap 200 is shown in place to protect against accidental damage to the needle assembly (not shown). FIG. 2 is a perspective view of the anesthetics needle device in FIG. 1.

FIG. 3 shows a perspective view of anesthetics needle device 100 of FIG. 2 with protective cap 200 being removed and thus exposing the needle assembly 500 having an exhaust needle 510 and a transfer needle 520. The anesthetics needle device 100 may also contain a buffer cartridge 300 and an exhaust cartridge 400. The buffer cartridge 300 is that device portion which contains a buffered solution (e.g., sodium bicarbonate) which is subsequently transferred into the anesthetic carpule for buffering.

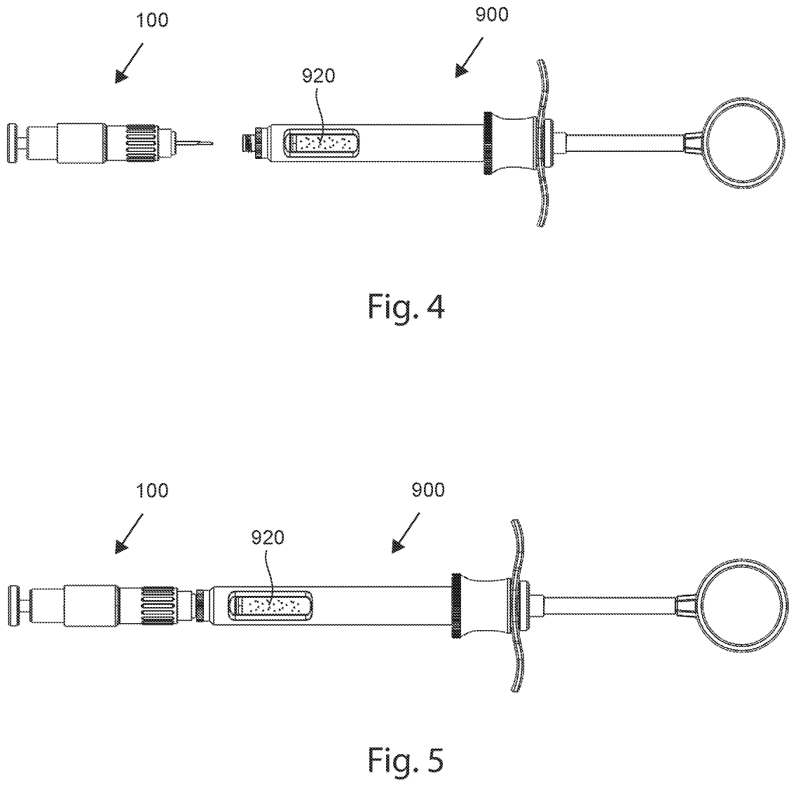

FIG. 4 shows a right-side elevational view of anesthetics needle device 100 being aligned with a syringe 900 having an anesthetic carpule 920 inside. It's contemplated that a medical professional can easily hold the anesthetics needle device 100 in one hand and the syringe 900 in another hand. Further, it's contemplated that the anesthetics needle device 100 may be used immediately prior to the subject medical procedure (e.g., painful dental procedure). In this way, the anesthetic does not need to be buffered several hours in advance (e.g., a batch process in the morning) and thus risk being discarded due to the limited shelf-life of buffered anesthetic.

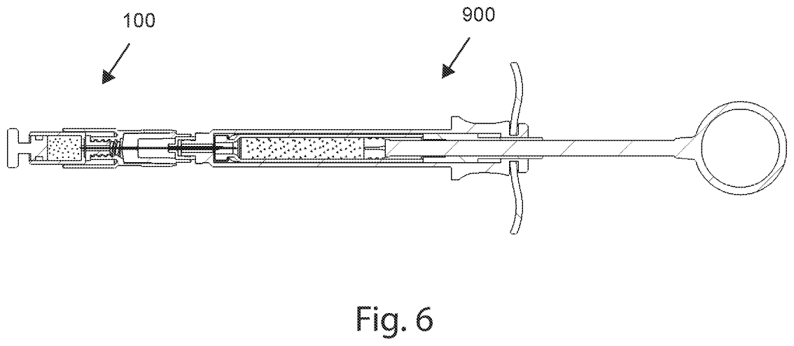

FIG. 5 shows a right-side elevational view of the anesthetics needle device 100 being inserted into the syringe 900 having an anesthetic carpule 920 inside. The anesthetics needle device 100 being adapted to easily and releasably-connect to syringe 900 and anesthetic carpule 920. FIG. 6 is a cross-sectional view of the anesthetics needle device 100, syringe 900 and anesthetic carpule 920 in FIG. 5.

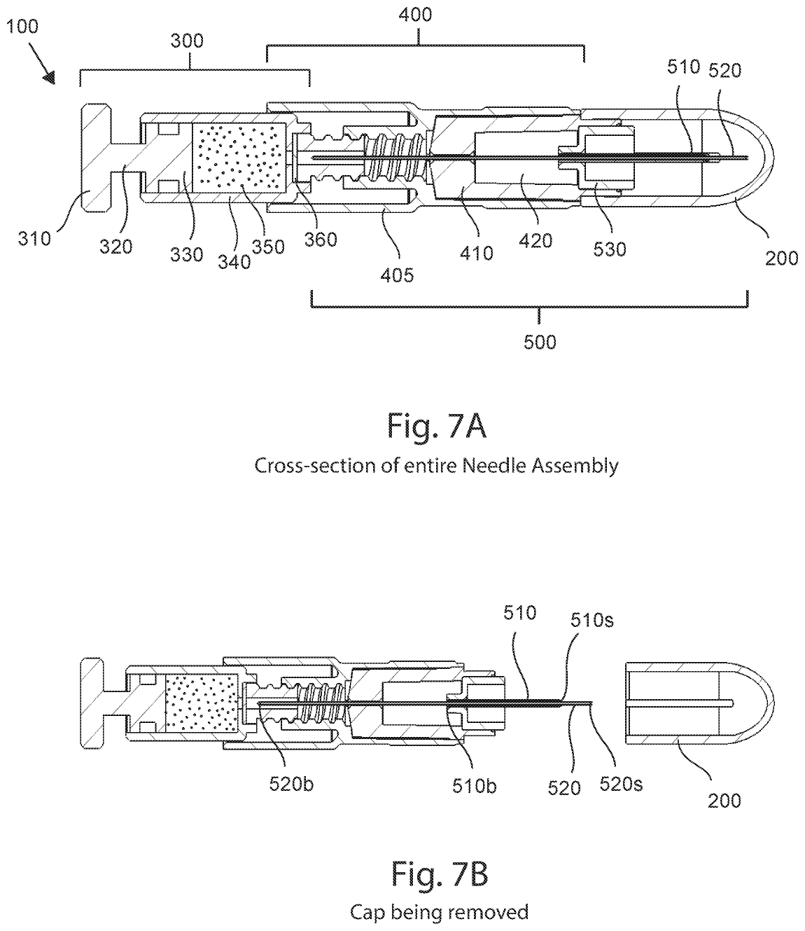

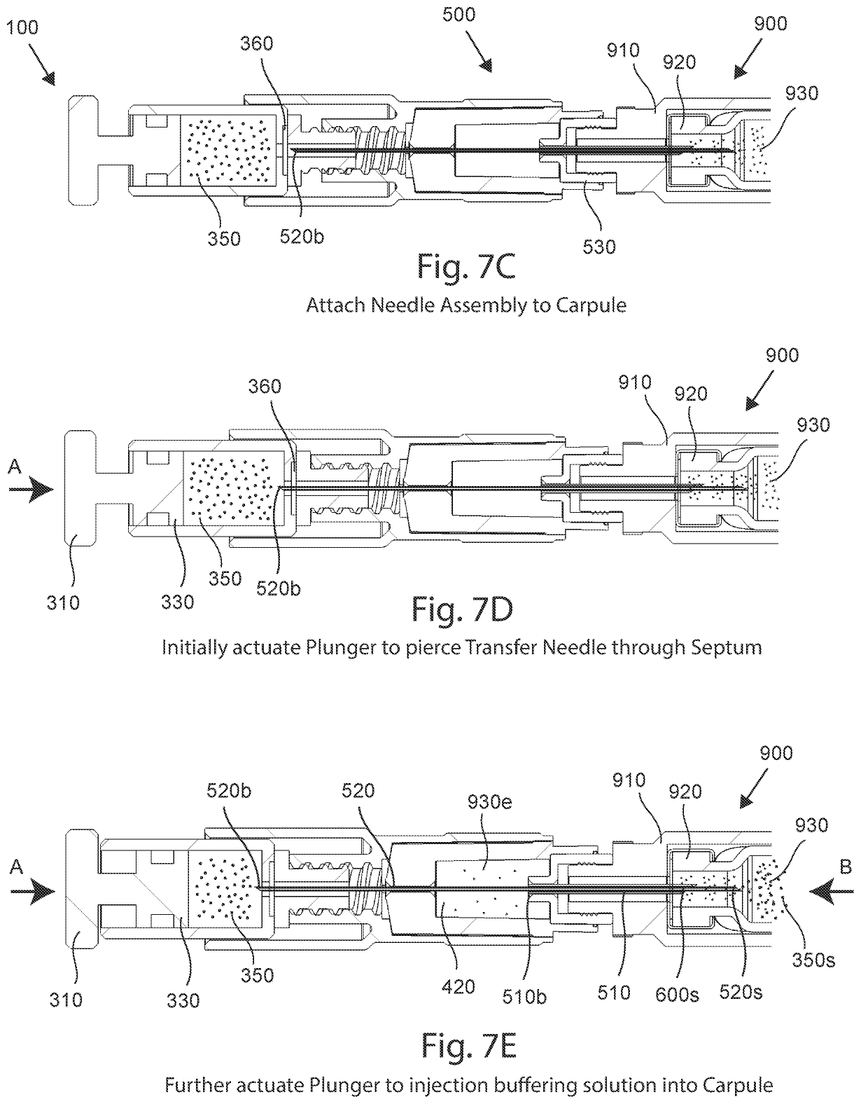

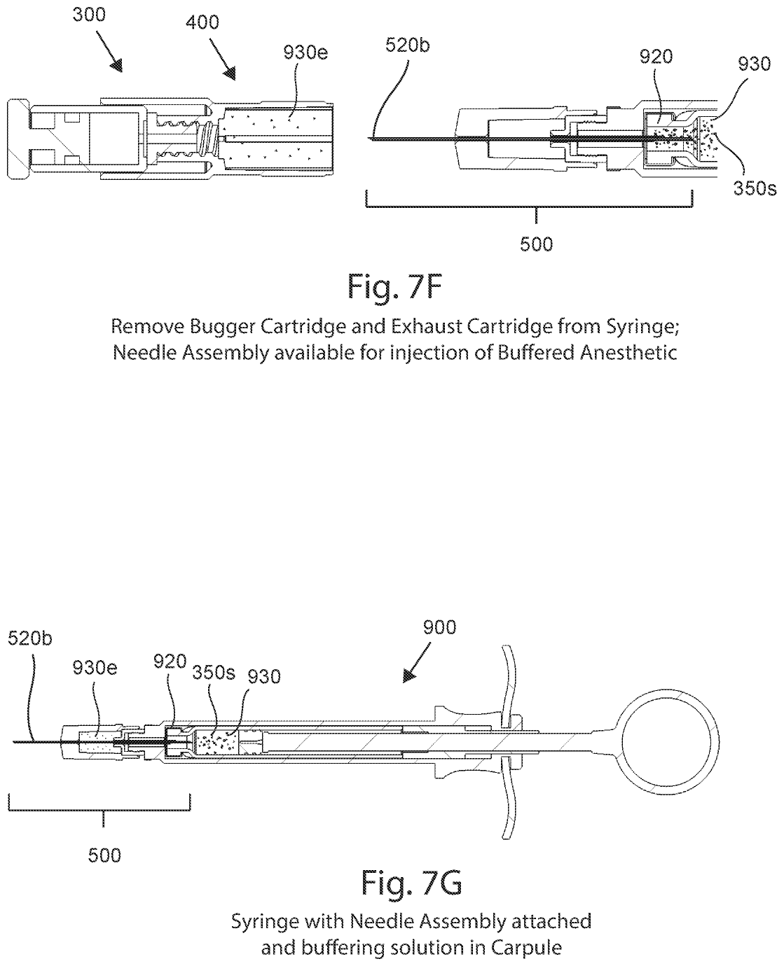

FIGS. 7A-7G provide a step-by-step pictorial description of how the anesthetic needle device 100 may be used in practice. FIG. 7A shows a cross-sectional view of the anesthetics needle device 100 in FIG. 2 wherein the protective cap 200 is still in place. In this view, the components of buffer cartridge are shown to include a plunger 310, plunger stem 320, piston 330 and buffer containment 340. The buffer containment 340 is adapted to store a buffer solution 350. A septum 360 is provided to keep the buffer solution stored and hermetically sealed until the device is ready to use. In this view, the components of exhaust cartridge 400 are shown to include an exhaust shell 405, exhaust housing 410 and an exhaust chamber 420. Before device 100 is used, it may be preferable for exhaust chamber 420 to be substantially empty. Exhaust chamber 420 is will be utilized in subsequent steps, more specifically, it's intended to collect anesthetic from the syringe that is displaced by buffer solution 350 as shown in later figures. In this view, an exhaust needle 510 and a transfer needle 520 are shown. In this non-limiting embodiment, said exhaust needle 510 and transfer needle 520 are configured to be coaxial. Additionally, transfer needle 520 extends further than exhaust needle 510 on both ends so as to perform the dual functions later described. The transfer and exhaust needles be structurally supported by connector 530. The connector 530 may also serve as a connection mechanism for releasably-engaging the anesthetics needle device 100 to syringe 900 and/or carpule 920, whichever type syringe-carpule combination is used by the medical professional. The exhaust needle 510, transfer needle 520 and connector 530 may collectively be referred to as needle assembly 500.

FIG. 7B shows a cross-sectional view of the anesthetics needle device 100 in FIG. 7A with the protective cap 200 being removed. At this point, the protective cap may either be discarded or kept for reapplication of the cap to anesthetics needle device 100 once the device is ready to be discarded. In this view, exhaust needle 510 is shown having a buffer-end 510b which is in close proximity to the buffer cartridge 300 and a syringe-end 510s which is in close proximity to syringe 900. Similarly, transfer needle 520 is shown having a buffer-end 520b which is in close proximity to the buffer cartridge 300 and a syringe-end 520s which is in close proximity to syringe 900.

FIG. 7C shows a cross-sectional view of the anesthetics needle device 100 in FIG. 7B with the needle assembly 500 being attached to syringe 900. In this step, the exhaust needle 510 and transfer needle 520 are inserted into syringe body 910 and pierce the carpule 920 to become in fluid communication with the anesthetic 930. In this step, it may be desirable for the transfer needle 520 to not yet pierce septum 360. In this way, the act of connecting to the syringe can be accomplished without concern of inadvertently injecting buffer solution.

FIG. 7D shows a cross-sectional view of the anesthetics needle device 100 in FIG. 7C with the plunger being initially actuated in the direction of arrow-A such that the transfer needle 520 pierces the septum 360 and becomes in fluid communication with the buffer solution 350. In the exemplary embodiment, the plunger may be actuated by pressing it forward which results in piston 330 to drive the movement of buffer solution 350 through transfer needle 520 into anesthetic carpule 920 to be mixed with anesthetic 930.

FIG. 7E shows a cross-sectional view of the anesthetics needle device in FIG. 7D with the plunger being further actuated in the direction of arrow-A such that buffer solution 350 is moved into the anesthetic carpule 920 and contemporaneously a portion of the anesthetic 930 is displaced through the exhaust needle 510 into the exhaust chamber 420. The anesthetic 930 which was displaced into exhaust chamber 420 may now be referred to as exhausted-anesthetic 930e. The exhaust chamber 420 may be adapted (e.g., having sufficient volume) to sufficiently accommodate a significant amount of anesthetic being displaced from the carpule 920 and into said exhaust chamber.

FIG. 7F shows a cross-sectional view of the anesthetic needle device 100 in FIG. 7E with the buffer cartridge 300 and exhaust cartridge 400 being removed and subsequently the needle assembly 500 being made available for injection of buffered anesthetic into the patient. FIG. 7G is a cross-sectional view of the needle assembly 500 in FIG. 7F showing the needle assembly being attached to the syringe 900 and the anesthetic carpule 920 now containing buffered anesthetic;

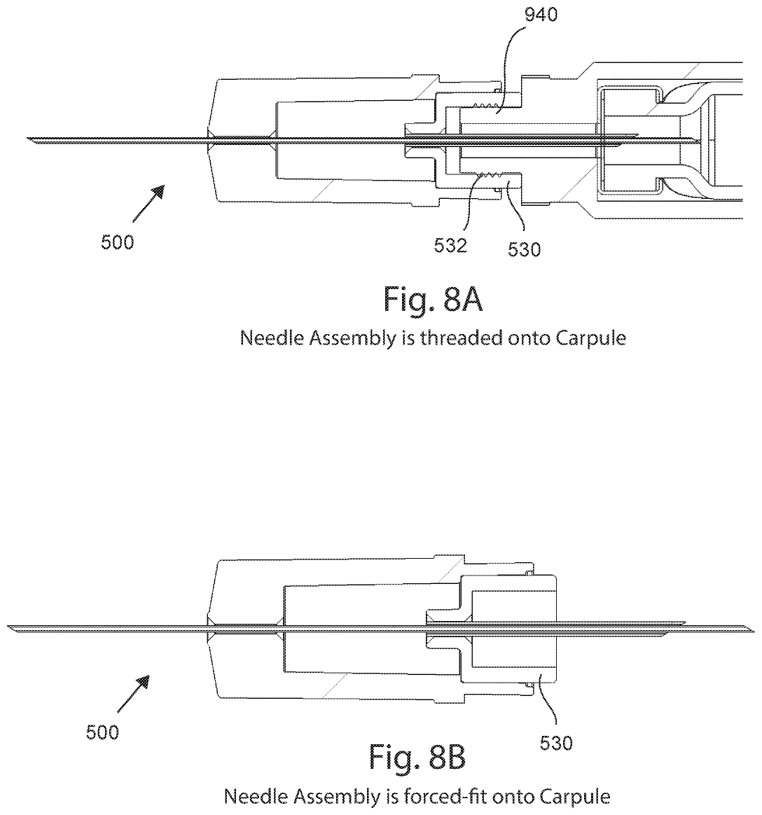

FIG. 8A shows a cross-sectional view of the needle assembly 500 and syringe nozzle 940 with the connector 530 being shown having threads 532 for secure releasable-attachment to the syringe. FIG. 8B is a cross-sectional view of an alternate needle assembly 500 with an alternate connector 530 being made of a rubber-lime material for force-fit attachment to the syringe. While keeping within the spirit of the present invention, one skilled in the art would appreciate that a variety of connection methods (e.g., simple adhesives or a locking mechanism) may be incorporated to releasably-engage the needle assembly to the syringe.

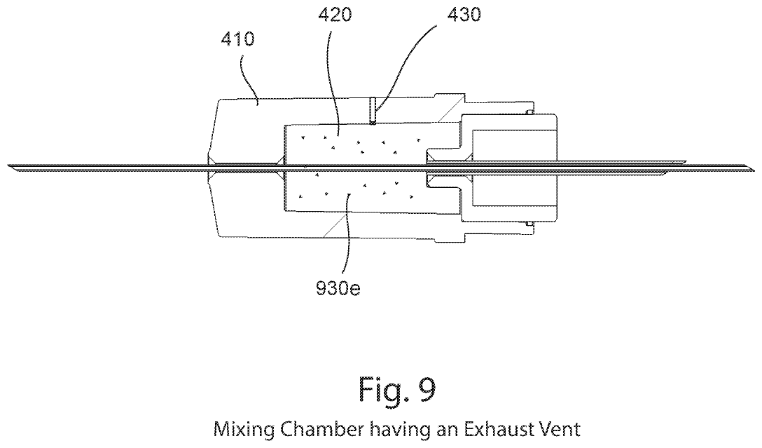

FIG. 9 shows a cross-sectional view of an alternate needle assembly 500 having an exhaust vent so that exhausted-anesthetic 930e may enter the mixing chamber 420 more easily. It may be desirable to have exhaust vent 430 to have one-way valve functionality so that molecules may only flow outwardly from mixing chamber 420.

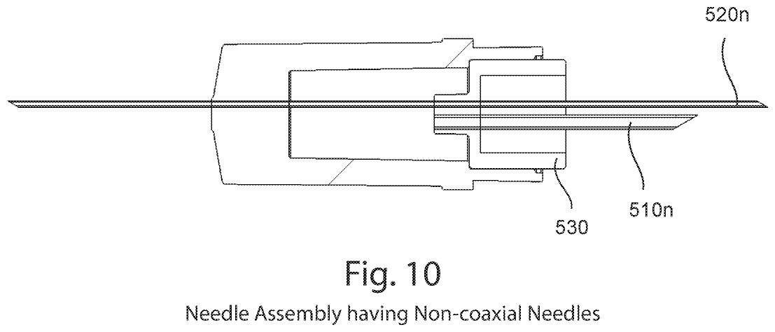

FIG. 10 shows a cross-sectional view of an alternate needle assembly wherein the transfer needle 520n and the exhaust needle 510n are non-coaxial. In this embodiment, it may be desirable to use a connector similar to FIG. 8B (e.g., rubber-like) wherein the needle assembly may be attached without rotational mechanics needed.

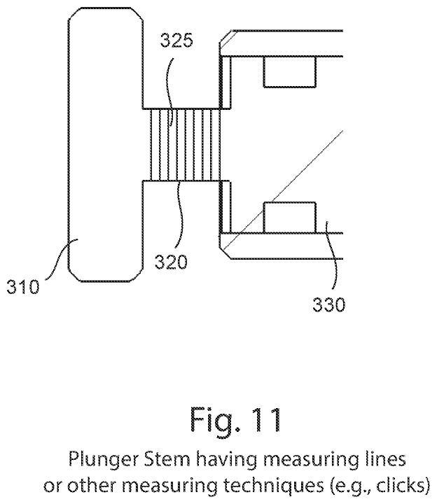

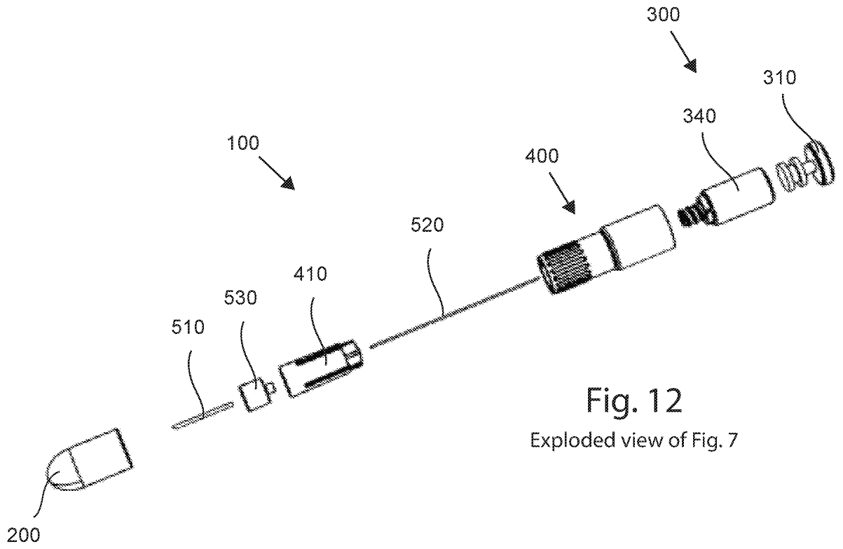

FIG. 11 shows a cross-sectional view of plunger 310 having plunger markings 325 on the plunger stem 320 so as assist in measuring the amount of buffered solution 350 being injected into anesthetic carpule 920. While keeping within the spirit of the present invention, one skilled in the art would appreciate that a variety of measuring methods may be incorporated to meter the desired amount of buffer solution 350 including the use of audible clicks, the use of mechanical clicks, the use of a rotational-dial that similarly pushes a piston and many other techniques.

FIG. 12 shows an exploded view of FIG. 7 showing an exemplary construction of the anesthetic needle device. Protective cap 200 is shown at the far-left end. In this exemplary embodiment, the exhaust needle 510 and transfer needle 520 are shown being coaxial. Further, the exhaust needle 510 is shown having a larger diameter than transfer needle 520 and is positioned outside of said transfer needle. Connector 530 is shown being inserted into exhaust housing 410 which sits within exhaust cartridge 400. Buffer cartridge 300 may be comprised of a buffer containment 340 for storing buffer solution (not shown) and subsequently transferred by actuation of the plunger 310.

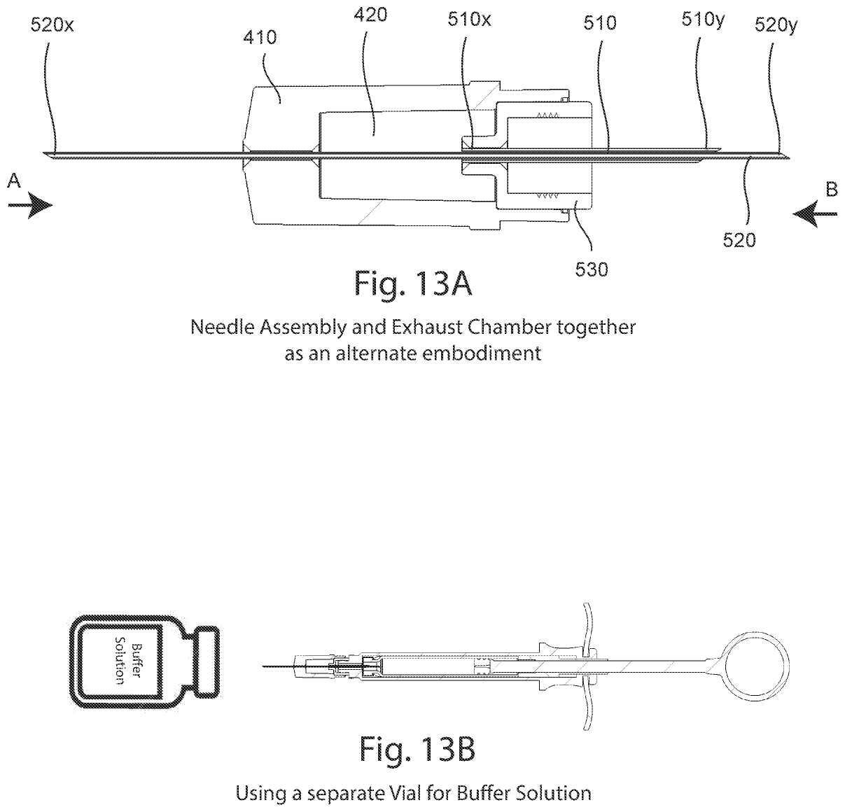

FIG. 13A shows a cross-sectional view of an alternate embodiment having only the needle assembly and exhaust chamber together. In this embodiment, the anesthetic needle device is capable of transferring a first solution into a syringe having a carpule containing a second solution and also injecting a mixture of the first and second solutions into a patient. The device having an exhaust housing 410 providing structural support for said device and also an exhaust chamber 420 being formed by the exhaust housing. The device also having a transfer needle 520 and an exhaust needle 510. The transfer needle 520 having a first-end 520x extending exterior to said exhaust housing 410 and a second-end 520y extending exterior to the exhaust needle 510. The transfer needle 520 being capable of transferring a first solution into the carpule containing a second solution. The exhaust needle 510 having a first-end 510x in communication with the exhaust chamber 420 and a second-end 510y extending short of the transfer needle second-end 520y. The exhaust needle 510 being capable of transferring a second solution out of the carpule and into the exhaust chamber 420. The device also including a connector 530 for providing structural support for transfer needle 520 and exhaust needle 510 and also providing releasable-attachment of the device to a syringe. With this particular embodiment, the present invention can be appreciated by merely attaching it to the syringe and perhaps introducing a buffer solution from a separate source (e.g., larger vial of buffer solution) as illustrated in FIG. 13B. This particular embodiment may be useful, for example, when multiple bufferings are needed and when multiple injections into the same patient may be anticipated. As with the earlier embodiments, this embodiment provides for the inward-transfer (arrow-A direction) of a buffer solution into a syringe (not shown), the displacement of the anesthetic by the incoming buffer solution and the subsequent outward-exhausting (arrow-B direction) of some anesthetic into an exhaust chamber 420. One skilled in the art would appreciate that the transfer needle and exhaust needle may be either coaxial or non-coaxial to each other.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

For example, one skilled in the art would appreciate that while buffer solution has been described that it may be advantageous to use other solutions for mixing into a carpule of anesthetic. Similarly, one skilled in the art would appreciate that while anesthetic has been described that other materials within a carpule may benefit from the introduction of other solutions.

For example, the exhaust needle and transfer needle may be constructed from sanitary metals, preferably stainless steel, or any other suitable material for the purposes contemplated herein.

For example, the majority of the components of the device may be constructed of a hard plastic or any other suitable material for the purposes contemplated herein.

For example, the present invention was described as being adapted to a durable syringe, likely made from steel tube or rolled metal; that said, the present invention may be adapted to work with any type of syringe generally used in the marketplace.

What is claimed is:

1. An anesthetic needle device comprising:
a buffer cartridge for storing and moving a buffer solution into a syringe having an anesthetic carpule;
an exhaust cartridge for transferring a buffer solution into a syringe having an anesthetic carpule and also receiving displaced anesthetic from the anesthetic carpule; and
a needle assembly for injecting a buffer solution into a syringe having an anesthetic carpule and also injecting a buffered-anesthetic into a patient.

2. The anesthetic needle device of claim 1 wherein said needle assembly comprises
a transfer needle, said transfer needle being capable of transferring a buffer solution from said buffer cartridge and into the anesthetic carpule;
an exhaust needle, said exhaust needle being capable of receiving the anesthetic which was displaced from the anesthetic carpule by the injection of the buffer solution; and
a connector, said connector providing structural support for said transfer needle and said exhaust needle and also providing releasable-attachment of said needle assembly to the syringe.

3. The anesthetic needle device of claim 2 wherein said transfer needle and said exhaust needle are coaxial to each other.

4. The anesthetic needle device of claim 2 wherein said transfer needle and said exhaust needle are non-coaxial to each other.

5. The anesthetic needle device of claim 3 wherein said exhaust cartridge comprises
an exhaust shell, said exhaust shell providing structural support for said exhaust cartridge; and
an exhaust housing adapted to form an exhaust chamber for the receiving of anesthetic which was displaced from the anesthetic carpule by the injection of the buffer solution;
wherein said transfer needle has a buffer-end and a syringe-end, said buffer-end being in fluid communication with buffer solution stored in the buffer cartridge, said syringe-end being in fluid communication with the anesthetic in the anesthetic carpule;
wherein said exhaust needle has a buffer-end and a syringe-end, said buffer-end being in fluid communication with exhaust chamber, said syringe-end being in fluid communication with the anesthetic in the anesthetic carpule.

6. The anesthetic needle device of claim 2 wherein said buffer cartridge comprises
a plunger for actuating the movement of buffer solution;
a plunger stem extending from said plunger;
a piston connected to said plunger stem and being adapted to push the buffer solution when actuated by said plunger; and
a buffer containment adapted to store the buffer solution and allowing for the movement of the piston.

7. The anesthetic needle device of claim 6 further comprising plunger markings on said plunger stem for assisting in the metering of the buffer solution.

8. The anesthetic needle device of claim 7 wherein the buffer cartridge further comprises a septum for providing a protective membrane of said buffer containment until such time that said transfer needle is repositioned to pierce said septum for subsequent moving of the buffer solution.

9. The anesthetic needle device of claim 2 further comprising a buffer solution stored in the buffer cartridge.

10. The anesthetic needle device of claim 2 wherein said connector has threads being adapted for releasable-engagement with the syringe.

11. The anesthetic needle device of claim 2 wherein said connector is made of a rubber-like material for the force-fit, releasable-engagement with the syringe.

12. An anesthetic needle device comprising:
a buffer cartridge for storing and moving a buffer solution into a syringe having an anesthetic carpule;
an exhaust cartridge for transferring a buffer solution into a syringe having an anesthetic carpule and also receiving displaced anesthetic from the anesthetic carpule;
a needle assembly for injecting a buffer solution into a syringe having an anesthetic carpule and also injecting a buffered-anesthetic into a patient; and
a buffer solution stored in the buffer cartridge.

13. The anesthetic needle device of claim 12 wherein said needle assembly comprises
a transfer needle, said transfer needle being capable of transferring a buffer solution from said buffer cartridge and into the anesthetic carpule;
an exhaust needle, said exhaust needle being capable of receiving the anesthetic which was displaced from the anesthetic carpule by the injection of the buffer solution; and
a connector, said connector providing structural support for said transfer needle and said exhaust needle and also providing releasable-attachment of said needle assembly to the syringe.

14. The anesthetic needle device of claim 13 wherein said transfer needle and said exhaust needle are coaxial to each other.

15. The anesthetic needle device of claim 13 wherein said transfer needle and said exhaust needle are non-coaxial to each other.

16. The anesthetic needle device of claim 14 wherein said exhaust cartridge comprises
an exhaust shell, said exhaust shell providing structural support for said exhaust cartridge; and
an exhaust housing adapted to form an exhaust chamber for the receiving of anesthetic which was displaced from the anesthetic carpule by the injection of the buffer solution;
wherein said transfer needle has a buffer-end and a syringe-end, said buffer-end being in fluid communication with buffer solution stored in the buffer cartridge, said syringe-end being in fluid communication with the anesthetic in the anesthetic carpule;
wherein said exhaust needle has a buffer-end and a syringe-end, said buffer-end being in fluid communication with exhaust chamber, said syringe-end being in fluid communication with the anesthetic in the anesthetic carpule.

17. The anesthetic needle device of claim 13 wherein said buffer cartridge comprises
a plunger for actuating the movement of buffer solution;
a plunger stem extending from said plunger;
a piston connected to said plunger stem and being adapted to push the buffer solution when actuated by said plunger; and
a buffer containment adapted to store the buffer solution and allowing for the movement of the piston.

18. An anesthetic needle device for transferring a first solution into a syringe having a carpule containing a second solution and also for injecting a mixture of the first and second solutions into a patient comprising:
an exhaust housing providing structural support for said device;
an exhaust chamber being formed by said exhaust housing;
a transfer needle, having a first-end extending exterior to said exhaust housing and a second-end extending exterior to the exhaust needle, said transfer needle being capable of transferring a first solution into the carpule containing a second solution;
an exhaust needle, having a first-end in communication with said exhaust chamber and a second-end extending short of said transfer needle second-end, said exhaust needle being capable of transferring a second solution out of the carpule and into said exhaust chamber; and
a connector providing structural support for said transfer needle and said exhaust needle and also providing releasable-attachment of said device to the syringe.

19. The anesthetic needle device of claim 18 wherein said transfer needle and said exhaust needle are coaxial to each other.

20. The anesthetic needle device of claim 18 wherein said transfer needle and said exhaust needle are non-coaxial to each other.
